(19) United States
(12) Patent Application Publication
(10) Pub. No.: US 2006/0036267 A1
(43) Pub. Date: Feb. 16, 2006

(54) Methods and apparatus for performing malabsorptive bypass procedures within a patient's gastro-intestinal lumen

(75) Inventors: Vahid Saadat, Saratoga, CA (US); Richard C. Ewers, Fullerton, CA (US); Ruey-Feng Peh, Sunnyvale, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: USGI Medical Inc., San Clemente, CA (US)

(21) Appl. No.: 10/916,768

(22) Filed: Aug. 11, 2004

(51) Int. Cl. A61B 17/08

(52) U.S. Cl. 606/153

(57) ABSTRACT

Methods and apparatus for performing malabsorptive bypass procedures within a patient's gastrointestinal lumen are described comprising, for example, gastroenterostomy procedures that are preferably performed in an endoscopic or laparoscopic fashion. Anastomosis between the patient's stomach and intestine allows food to bypass at least a portion of the patient's stomach and/or intestine, thereby providing a malabsorptive region. The malabsorptive procedure may be accompanied by additional procedures, for example, pyloric occlusion, pyloroplasty, gastroplasty, gastric tissue destruction and/or intestinal pleating.

### BACKGROUND OF THE INVENTION

Field of the Invention

The present invention relates to methods and apparatus for performing a malabsorptive bypass procedure within a patient's gastro-intestinal (“GI”) lumen. More particularly, the present invention provides methods and apparatus for performing gastroenterostomy procedures, preferably in an endoscopic or laparoscopic fashion.

Extreme or morbid obesity is a serious medical condition pervasive in the United States and other countries. Its complications include hypertension, diabetes, coronary artery disease, stroke, congestive heart failure, multiple orthopaedic problems and pulmonary insufficiency with markedly decreased life expectancy.

Several surgical techniques have been developed to treat morbid obesity, including bypassing an absorptive surface of the small intestine, bypassing a portion of the stomach and reducing the stomach size, e.g., via Vertical Banded Gastroplasty (“VBG”) or Magenstrasse and Mill. These procedures may be difficult to perform in morbidly obese patients and/or may present numerous potentially life-threatening post-operative complications. Thus, less invasive techniques have been pursued.

U.S. Pat. Nos. 4,416,267 and 4,485,805 to Garren et al. and Foster, Jr., respectively, propose disposal of an inflated bag within a patient's stomach to decrease the effective volume of the stomach that is available to store food. Accordingly, the patient is satiated without having to consume a large amount of food. A common problem with these inflated bags is that, since the bags float freely within the patient's stomach, the bags may migrate to, and block, a patient's pyloric opening, the portal leading from the stomach to the duodenum, thereby restricting passage of food to the remainder of the gastro-intestinal tract.

Apparatus and methods also are known in which an adjustable elongated gastric band is disposed around the outside of a patient's stomach near the esophagus to form a collar that, when tightened, squeezes the stomach into an hourglass shape, thereby providing a stoma that limits the amount of food that a patient may consume comfortably. An example of an adjustable gastric band is the LAP-BAND® made by INAMED Health of Santa Barbara, Calif.

Numerous disadvantages are associated with using the adjustable gastric band. First, the band may be dislodged if the patient grossly overeats, thereby requiring additional invasive surgery to either reposition or remove the band. Similarly, overeating may cause the band to injure the stomach wall if the stomach over-expands. The laparoscopic disposal of the gastric band around the stomach requires a complex procedure, requires considerable skill on the part of the clinician, and is not free of dangerous complications.

In view of the drawbacks associated with prior art techniques for treating morbid obesity, it would be desirable to provide improved methods and apparatus for performing malabsorptive bypass procedures within a patient's gastro-intestinal lumen.

### BRIEF SUMMARY OF THE INVENTION

Improved methods and apparatus for performing malabsorptive bypass procedures within a patient's gastro-intestinal (“GI”) lumen are achieved by providing methods and apparatus for performing gastroenterostomy procedures within the lumen, preferably in an endoscopic or laparoscopic fashion. In one variation, a steerable and/or shape-lockable instrument may be advanced through the patient's stomach, pylorus and duodenum to the patient's jejunum. Once positioned within the jejunum, alignment mechanisms, such as light, telemetry, imaging, sensing, magnetism, steering, mechanical steering, shape-locking and/or rigidizing may be utilized to align the instrument and a portion of the jejunum adjacent with the patient's stomach. One or more securing elements then may be utilized to secure the patient's stomach to the adjacent portion of jejunum. The securing elements may lead to pressure necrosis and adjacent healing of tissue between the stomach and the jejunum, thereby forming a side-to-side anastomosis between the stomach and the jejunum and achieving gastro-jejunostomy.

Anastomosis alternatively may be achieved by creating a puncture between the patient's intestine and stomach. Edges of the puncture may be sealed via securing elements. Anastomosis between the patient's stomach and intestine allows food to bypass at least a portion of the patient's stomach and/or intestine, thereby providing a malabsorptive region within the patient's GI lumen.

Malabsorptive GI procedures may be accompanied by additional procedures. For example, an occlusive procedure may be performed to partially or completely close down the pylorus, thereby preventing or reducing the flow of food through the pylorus. This may be achieved by causing inflammation within the pylorus, i.e. pyloritis, or by forming stricture, embolization or stenosis within the pylorus, e.g. pyloristenosis. Inflammation may, for example, be achieved via chemical irritants, radiofrequency (“RF”) irradiation, heating, burning, etc. Stenosis may, for example, be achieved via bulking agents injected into the wall of the pylorus. As yet another alternative, the pylorus may be sutured or otherwise shut mechanically, e.g., via adhesives, hydrogels or inflatable balloons.

As an alternative to occluding the patient's pylorus, it may be desirable to perform pyloroplasty to render the patient's pyloric sphincter incompetent. This may be achieved, for example, using a balloon catheter to dilate the pylorus. Additional techniques include, for example, injecting agents into the pyloric sphincter that render the sphincter incompetent, or stimulating the sphincter with RF radiation.

In addition or as an alternative to procedures performed on the patient's pylorus, gastroplasty procedures may be performed on the patient's stomach, e.g., restrictive procedures. For example, the patient's gastric lumen may be partitioned to reduce an effective cross-sectional area of the lumen and restrict the passage of food therethrough. Furthermore, at least a portion of the tissue within the gastric lumen may be destroyed or otherwise reduced. Tissue destruction may be achieved, for example, with RF, plasma or other energy sources. When performed in conjunction with partitioning, tissue in the excluded portion of the patient's stomach may be destroyed.

In addition to the mentioned procedures, plications may be formed and secured that encompass the walls of both the patient's small intestine and stomach. Furthermore, a section of the patient's small intestine may be pleated or otherwise bunched up, and secured to the patient's stomach, e.g., proximal of an ostomy between the stomach and the small intestine. Additional procedures will be apparent to those of skill in the art.

### BRIEF DESCRIPTION OF THE DRAWINGS

FIGS. 1A-1D are schematic side views, partially in section, illustrating exemplary apparatus and methods for performing gastroenterostomy procedures.

FIGS. 2A-2C are schematic detail views, partially in side- or cross-section, illustrating exemplary securing element(s) for securing a patient's stomach to the patient's intestine at a desired location to form or maintain an ostomy therebetween.

FIG. 3 is a schematic detail view, partially in section, of an alternative securing element in use within the patient's GI lumen.

FIG. 4 is a schematic detail sectional view of another alternative securing element in use within the GI lumen.

FIG. 5 is a schematic detail sectional view of yet another alternative securing element in use within the GI lumen.

FIGS. 6A-6C are schematic side views, partially in section, illustrating apparatus and methods for at least partially occluding the patient's pylorus.

FIGS. 7A and 7B are, respectively, a schematic side-sectional view and an enlarged cross-sectional view along section line A-A of FIG. 7A, illustrating alternative methods and apparatus for at least partially occluding the pylorus.

FIGS. 8A and 8B are schematic side views, partially in section, illustrating apparatus and methods for performing gastroplasty within the patient's stomach.

FIGS. 9A and 9B are schematic side views, partially in section, illustrating apparatus and methods for destroying tissue within the patient's stomach.

FIGS. 10A-10C are schematic side views, illustrating variations of steerable/shape-lockable overtubes for performing gastroenterostomy procedures.

FIG. 11 is a schematic side view, partially in section, illustrating a method of using the overtube of FIG. 10 to align a portion of a patient's jejunum with the patient's stomach, thereby facilitating gastroenterostomy procedures.

FIGS. 12A and 12B are schematic detail side views, partially in section, illustrating a method of performing gastroenterostomy.

FIGS. 13A and 13B are schematic detail side views, partially in section, illustrating another method of performing gastroenterostomy.

FIGS. 14A and 14B are schematic detail side views, partially in section, illustrating a method of securing a patient's stomach to the patient's intestine at a desired location.

FIGS. 15A-15C are schematic side views, partially in section, illustrating a method of pleating a patient's intestine for conducting a malabsorptive procedure.

### DETAILED DESCRIPTION OF THE INVENTION

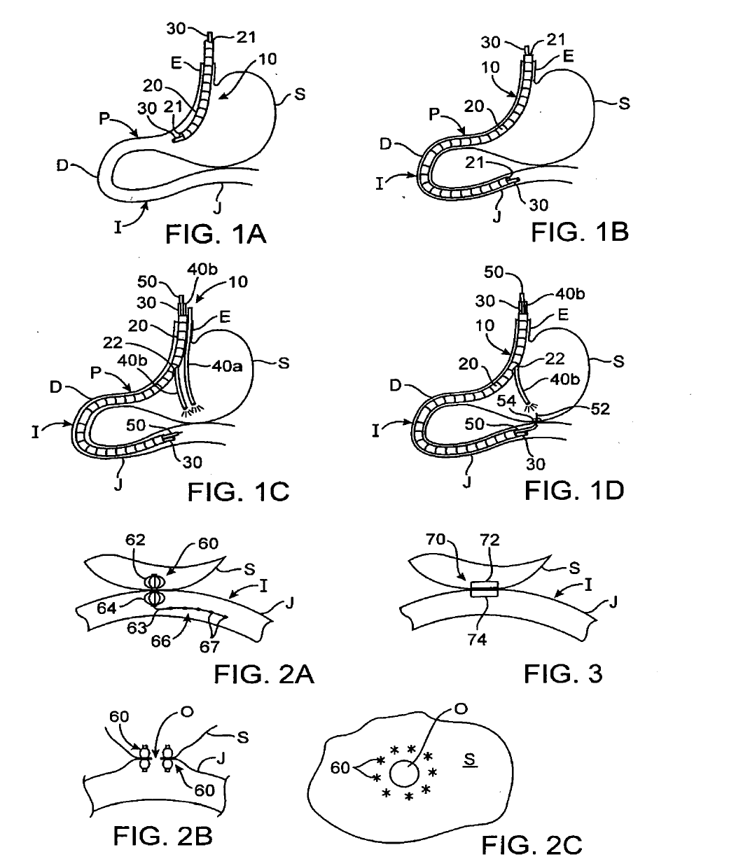

With reference to FIG. 1, illustrative methods and apparatus for performing malabsorptive bypass procedures within a patient's gastro-intestinal (“GI”) lumen are described. In FIG. 1A, apparatus 10, comprising steerable and/or shape-lockable/rigidizable overtube 20, has been advanced down a patient's esophagus E into the patient's stomach S. Applicant has previously described steerable and rigidizable overtubes, for example, in co-pending U.S. patent application Ser. No. 10/797,485, filed Mar. 9, 2004, which is incorporated herein by reference in its entirety.

Overtube 20 preferably comprises one or more lumens 21 through which additional diagnostic or therapeutic instruments may be advanced. Endoscope 30 may be disposed within a lumen 21 to provide visual feedback during steering of overtube 30 through the patient's GI lumen. As seen in FIG. 1B, the overtube may be advanced through the patient's stomach S, past the pylorus P, into the small intestine I. Once disposed within the small intestine, overtube 20 may be utilized to perform a laparoscopic or endoscopic gastroenterostomy procedure. Furthermore, the overtube may be shape-locked or rigidized to maintain its orientation.

In FIG. 1B, overtube 20 illustratively has been advanced past duodenum D into jejunum J for performing a gastro-jejunostomy procedure. In FIG. 1C, an alignment mechanism is utilized to align anastomosis instruments that are disposed in the patient's jejunum, e.g., within a lumen 21 of overtube 20 and/or within a working lumen of endoscope 30, with the patient's stomach. The alignment mechanism illustratively comprises light source(s) 40, e.g. fiber optic light source(s), advanced through the patient's esophagus E into the patient's stomach S. Light source 40a illustratively has been advanced adjacent to overtube 20, while light source 40b illustratively has been advanced through a lumen 21 of the overtube and out through an optional side port 22.

As will be apparent, any number of light sources, including a single light source, may be provided and advanced in any desired manner. Once the light source is positioned within the patient's stomach, alignment is achieved by shining light through source 40 and visualizing or otherwise measuring an increase in light intensity with instruments disposed within the jejunum. Overtube 20, endoscope 30 and/or the anastomosis instruments disposed within jejunum J, may be rotated, steered, shape-locked, etc., to align the instruments with the region of enhanced light, and thereby align the portion of jejunum J adjacent to the patient's stomach S.

Once properly aligned, anastomosis instruments 50 may be utilized to perform the malabsorptive bypass procedure. In FIG. 1D, coring needle 52 has pierced the walls of jejunum J and stomach S, thereby forming a passageway between the two organs. A securing element, such as suture 54, may be utilized to maintain the passageway and complete the side-to-side anastomosis, thereby endoluminally achieving gastro-jejunostomy. Suture 54 may, for example, be applied endoluminally through anastomosis instrument 50 or any other endoluminal or laparoscopic suturing instrument(s) to approximate the tissue. Overtube 20, endoscope 30, light source alignment mechanism 40 and anastomosis instruments 50 then may optionally be removed from the patient, thereby completing the procedure.

Upon completion of the procedure, food ingested by the patient may bypass a portion of the patient's stomach, as well as a section of the intestine, by directly draining into the intestine through the ostomy formed between the stomach and the jejunum. This may reduce calories absorbed by the bypassed section, thereby contributing to weight loss. The bypassed section optionally may be excluded completely from the patient's GI lumen, as described hereinafter.

In FIG. 1, alignment illustratively was achieved via a light source. However, it should be understood that alternative alignment mechanisms may be used including, but not limited to, telemetry, imaging, sensing, mechanical steering, magnetism and combinations thereof. Additional alignment mechanisms will be apparent to those of skill in the art.

Referring now to FIG. 2, an additional exemplary securing element for securing the patient's stomach to the patient's intestine is described. Securing element 60 comprises stomach anchor 62 and intestinal anchor 64 connected by suture 63. Anchors 62, 64 may comprise, e.g., expandable basket-type anchors, delivered and deployed through overtube 20. One or more stomach anchors 62 may be advanced in a low profile configuration from the jejunum and into the stomach S where the one or more anchors may be expanded to prevent withdrawal back through the tissue. The intestinal anchor(s) 64 may be deployed and expanded within the jejunum J in apposition to the stomach anchor(s) 62. Once both anchors 62, 64 have been expanded, suture 63 connecting them may be tensioned to draw the anchors 62, 63 towards one another, thereby drawing the portion of stomach S and jejunum J adjacent to one another. Additional anchor securing elements, including methods for placing the elements, are described, for example, in Applicant's co-pending U.S. patent application Ser. No. 10/840,950, filed May 7, 2004, which is incorporated herein by reference in its entirety.

In contrast to the gastroenterostomy procedure of FIG. 1, securing element 60 of FIG. 2A may form an anastomosis between stomach S and jejunum J via pressure necrosis. Stress imposed by element 60 on the walls of the stomach and the jejunum may be greater than blood perfusion pressure within the walls, thereby locally starving the wall tissue of blood and causing element 60 to erode through the tissue and harmlessly pass through the patient. This concurrently initiates a wound healing response at the edge of the eroded tissue that fuses the stomach to the jejunum, while leaving an ostomy between the two organs.

Securing element 60 optionally may comprise weight 66 that is connected, for example, to intestinal anchor 64 or suture 63. Weight 66 may comprise a discrete element or may be distributed over a series of elements 67, as in FIG. 2A. Distributing the weight over a series of elements is expected to reduce a risk of intestinal occlusion due to the weight. Weight 66 is expected to accelerate pressure necrosis of tissue disposed between anchors 62 and 64 by increasing the stress imposed on the tissue, as well as by providing cyclically increased loads as food passing through intestine I tugs on weight 66.

As an alternative, or in addition, to their use in forming a gastroenterostomy via pressure necrosis, anchor securing elements like element 60 may be used to maintain an ostomy. When maintaining an ostomy, the anchor securing elements preferably apply a tissue stress that is less than blood perfusion pressure within the tissue, thereby reducing a risk of pressure necrosis. For example, as seen in side- and cross-section in FIGS. 2B and 2C, a plurality of anchor securing elements 60 may be placed in a ring around ostomy O. The ostomy may be formed as described previously with respect to FIG. 1, or may otherwise be dilated or incised out. The securing elements may be used in place of (or in combination with) suture 54 of FIG. 1D to maintain the ostomy. Furthermore, the securing elements may be placed before, after or during formation of ostomy O.

Referring now to FIG. 3, another alternative securing element is described that utilizes magnetic attraction. Mating magnetic elements for pressure necrosis anastomosis previously have been described, for example, in U.S. Pat. No. 5,690,656 to Cope et al., and U.S. Pat. No. 6,558,400 to Deem et al., both of which are incorporated herein by reference in their entireties. In FIG. 3, securing element 70 comprises stomach magnet 72 and intestinal magnet 74. Alignment and placement of element 70 may be achieved, for example, by advancing intestinal magnet 74 into jejunum J, e.g., via steerable and/or rigidizable overtube 20. Stomach magnet 72 then may be mated with the intestinal magnet by placing the stomach magnet in the patient's stomach S and allowing magnetic attraction to draw the stomach and intestinal magnets together. Gastroenterostomy then may be achieved via pressure necrosis between the magnets of element 70.

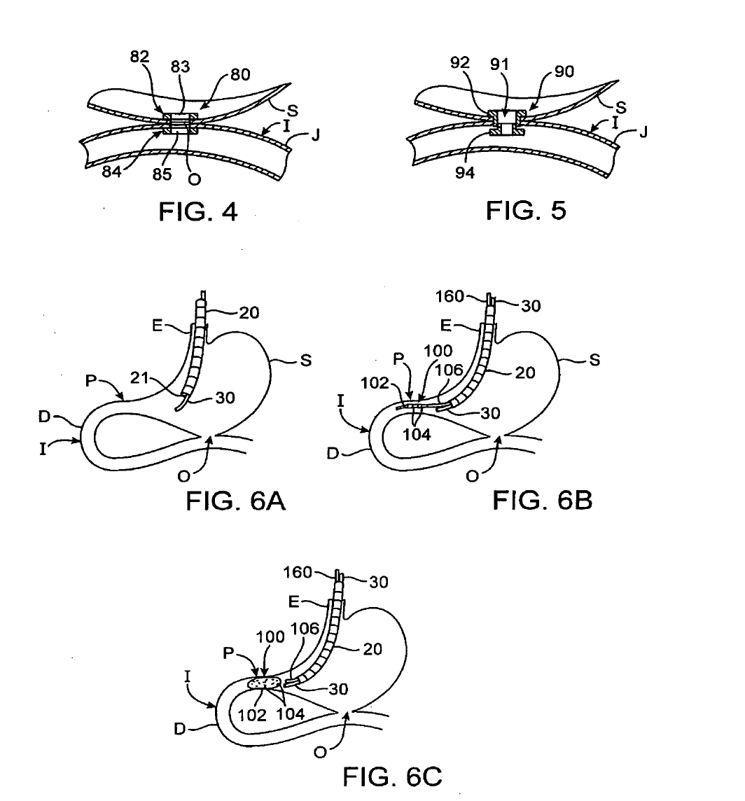

Referring now to FIG. 4, an alternative magnetic securing element is described. In FIG. 4, securing element 80 comprises stomach washer magnet 82 and intestinal washer magnet 84. Magnets 82 and 84 comprise central openings 83 and 85, respectively. As seen in FIG. 4, once magnets 82 and 84 have been magnetically attached, ostomy O may be formed through central openings 83 and 85, e.g., via coring needle 52 of anastomosis apparatus 50 of FIG. 1, thereby completing gastroenterostomy. Optionally, the ostomy may be formed prior to placement of magnets 82 and 84, and securing element 80 may be used to maintain the ostomy. A magnitude of magnetic attraction exerted between magnets 82 and 84 may be specified as desired, for example, to achieve pressure necrosis, or to provide for long-term maintenance of the securing element 80 within the patient.

Referring now to FIG. 5, another alternative securing element is described. Element 90 comprises a two-piece mating rivet having an optional central opening 91 that forms an ostomy between stomach S and intestine I. Stomach piece 92 and intestinal piece 94 may be magnetically attracted and/or may be mechanically mated. Furthermore, stress applied to surrounding tissue by element 90 may yield pressure necrosis or may provide for long-term maintenance of the element across the ostomy. Element 90 optionally may be bioabsorbable or bioresorbable.

With reference to FIG. 6, apparatus and methods for at least partially occluding a patient's pylorus are described. In FIG. 6, a gastroenterostomy procedure has already been performed to provide ostomy O between stomach S and intestine I. However, it should be understood that complete or partial occlusion of the pylorus optionally may be performed prior to (or without) formation of the gastroenterostomy.

As seen in FIG. 6A, overtube 20 has been advanced (or retracted from intestine I) into the patient's stomach S. With the outlet of the overtube positioned in proximity to pylorus P, and under optional visual guidance provided by endoscope 30, occlusive element 100 is advanced out of a lumen 21, such that it is coaxially disposed within the pyloric opening, as seen in FIG. 6B. Occlusive element 100 may, for example, comprise a water-swellable hydrogel, an adhesive, etc. In FIG. 6, the occlusive element illustratively comprises inflatable balloon 102 having barbs 104 detachably coupled to inflation catheter 106.

As seen in FIG. 6C, the inflatable balloon may be expanded into contact with the wall of pylorus P, e.g., via inflation catheter 106, such that barbs 104 irreversibly engage the wall and maintain the balloon within the pylorus, thereby occluding the pylorus. Balloon 102 then may be decoupled from catheter 106.

Occlusion of the pylorus may, for example, completely exclude the section of intestine I between the pylorus and gastroenterostomy O, e.g., completely exclude duodenum D. Such exclusion may further reduce absorption of calories while food travels through the patient's GI lumen.

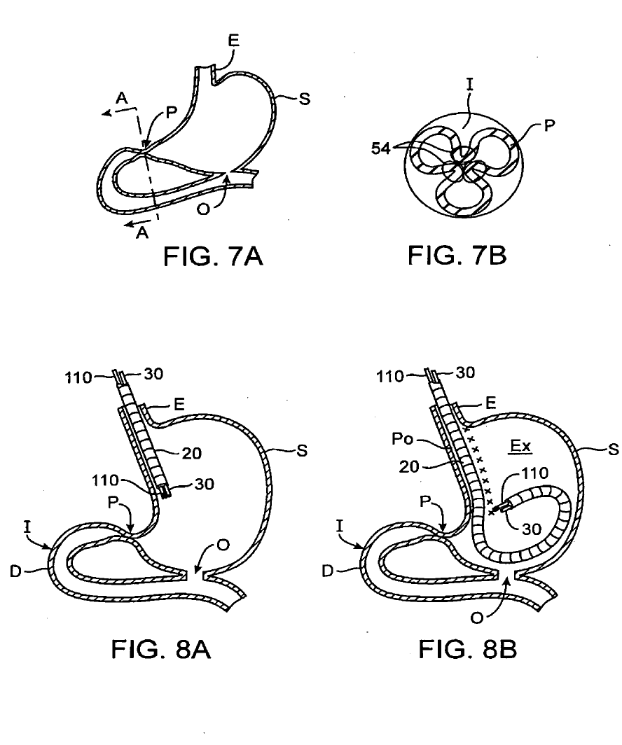

Referring to FIG. 7, alternative methods and apparatus for occluding a patient's pylorus are described. As seen in FIG. 7, suture 54 has been routed about the circumference of pylorus P and then drawn down and tied off to approximate the walls of the pylorus, thereby at least partially occluding the pylorus. As an alternative to using suture 54, securing elements, such as securing elements 60 of FIG. 2, may be utilized to at least partially occlude pylorus P.

In addition, or as an alternative, to the pyloric occlusion techniques already discussed, occlusion optionally may be achieved by causing inflammation within the pylorus, i.e. pyloritis, or by forming stricture, embolization or stenosis within the pylorus, e.g. pyloristenosis. Inflammation may, for example, be achieved via chemical irritants, radiofrequency (“RF”) irradiation, heating, burning, etc. Stenosis may, for example, be achieved via bulking agents injected into the wall of the pylorus.

As an alternative to occluding the patient's pylorus, it may be desirable to perform a pyloroplasty procedure to render the patient's pyloric sphincter incompetent. This may be achieved, for example, using a balloon catheter to dilate the pylorus. Additional techniques include, for example, injecting agents into the pyloric sphincter that render it incompetent or stimulating the sphincter with RF radiation.

Referring now to FIG. 8, illustrative apparatus and methods for performing gastroplasty within the patient's stomach are described. In FIG. 8, a gastroenterostomy procedure has already been performed, and the patient's pylorus has been occluded. However, it should be understood that gastroplasty optionally may be performed without performance of gastroenterostomy and/or pyloric occlusion.

As seen in FIG. 8A, overtube 20 has been advanced (or retracted) to a position within stomach S whereby the overtube is disposed in proximity to the stomach's lesser curvature. In FIG. 8B, overtube 20 has been steered, shape-locked and otherwise manipulated to position gastroplasty instruments 110 for forming, approximating and securing anterior and posterior tissue folds along a length of the stomach to partition the stomach into pouch Po and excluded region Ex, thereby achieving gastroplasty. Overtube 20 and gastroplasty apparatus 110, as well as optional endoscope 30, optionally then may be removed from the patient to complete the procedure.

Applicant has previously described methods and apparatus for achieving gastroplasty, for example, in U.S. patent application Ser. No. 10/841,415 (Attorney Docket No. 021496-000800), filed May 7, 2004; Ser. No. 10/841,233 (Attorney Docket No. 021496-001400), filed May 7, 2004, and Ser. No. 10/734,562, filed Dec. 12, 2003; all of which are incorporated herein by reference in their entireties. Any of the methods and apparatus described therein additionally or alternatively may be utilized to perform gastroplasty.

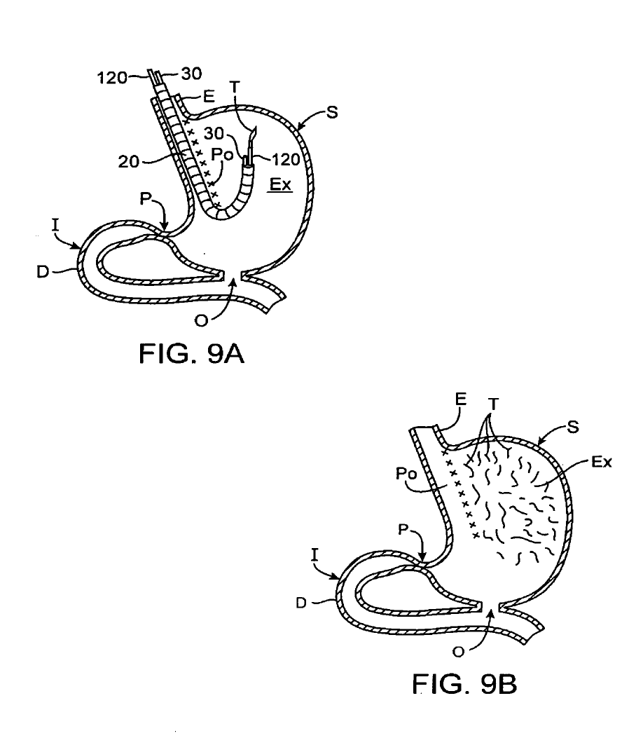

Referring now to FIG. 9, illustrative apparatus and methods for destroying tissue within the patient's stomach are described. In FIG. 9, gastroenterostomy and gastroplasty procedures have already been performed, and the patient's pylorus has been occluded. However, it should be understood that tissue destruction optionally may be performed without performance of gastroenterostomy, gastroplasty and/or pyloric occlusion. Tissue destruction, gastroenterostomy, gastroplasty and pyloric occlusion procedures (as well as any other procedures) may be performed in any combination, with any subset of the procedures and/or in any order, as desired.

As seen in FIG. 9A, a distal end of overtube 20 may be positioned, for example, within excluded region Ex of stomach S. Tissue destruction instruments 120 then may be advanced through a lumen 21 of the overtube and actuated to locally destroy or otherwise reduce, make incompetent, etc., gastric tissue T. Tissue destruction instruments 120 may comprise, for example, RF, plasma, electrocautery, cryoablation, Argon plasma coagulation, mechanical abrasion, combinations thereof, and/or other energy source instruments. As seen in FIG. 9B, tissue destruction may be achieved at multiple locations, e.g., within excluded region Ex, and then overtube 20 and destruction instruments 120 may be removed from the patient.

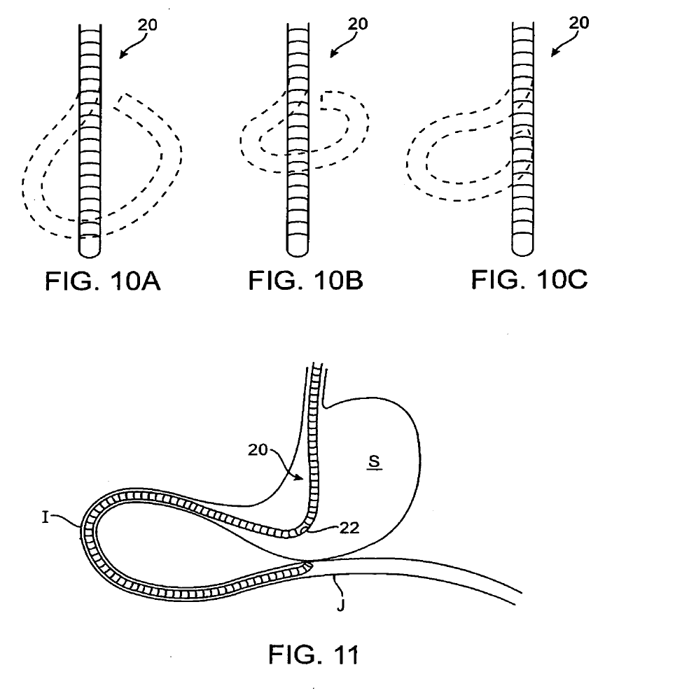

With reference now to FIGS. 10-15, additional methods and apparatus for performing malabsorptive gastro-intestinal procedures are described. It should be understood that any of the methods and apparatus described therein may be utilized in combination with any of the methods and apparatus described previously, and vice versa.

Referring to FIG. 10, variations of steerable/shape-lockable overtubes for performing gastroenterostomy procedures are described. As mentioned earlier, Applicant has previously described steerable and/or shape-lockable overtubes, for example, in co-pending U.S. patent application Ser. No. 10/797,485, filed Mar. 9, 2004, which has been incorporated herein by reference. As described therein, steering and rigidizing of an overtube may, for example, be achieved via tensionable wires disposed within or along the overtube.

FIGS. 10A-10C illustrate variations of overtube 20, wherein the overtube is steerable/rigidizable to a pre-determined shape or configuration. As seen in dotted profile, the overtube of FIG. 10A is steerable and/or shape-lockable to a retroflexed configuration about a longitudinal axis of overtube 20. In FIG. 10B, overtube 20 may be retroflexed with a tighter radius of curvature. In FIG. 10C, the overtube may be retroflexed to a position off-axis from the longitudinal axis of the overtube. In all of FIG. 10, an outlet distal end of overtube 20 is aligned with the body of the overtube in the retroflexed configurations. Additional/alternative steered configurations for overtube 20 will be apparent to those of skill in the art.

With reference to FIG. 11, an overtube 20 in accordance with FIG. 10 may be advanced through a patient's stomach S into the patient's jejunum J. The overtube then may be steered/rigidized to a retroflexed configuration that aligns an outlet distal end of the overtube within the patient's jejunum with a body of the overtube disposed in the patient's stomach, thereby aligning and/or approximating a portion of the patient's jejunum with the patient's stomach. In FIG. 11, overtube 20 comprises optional side port 22, and the distal end of overtube 20 is aligned with the side port.

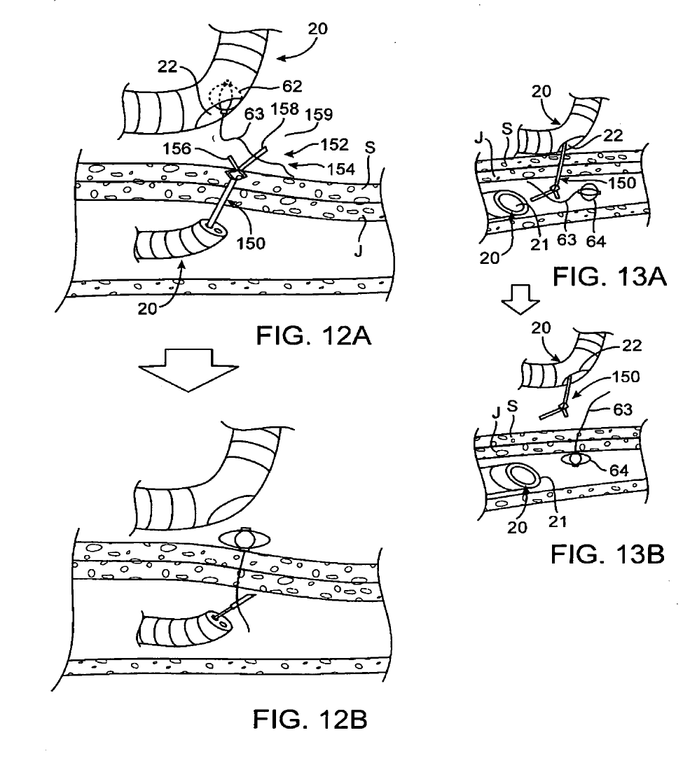

Referring now to FIG. 12, with the patient's stomach and jejunum aligned/approximated via overtube 20, a gastroenterostomy procedure may be performed. In FIG. 12A, previously-described stomach anchor 62 of securing element 60 of FIG. 2 is disposed within overtube 20 in proximity to side port 22. Suture 63 extends out of the side port and may be grasped by combination needle and grasper apparatus 150. Applicant has previously described apparatus 150, for example, in co-pending U.S. patent application Ser. No. 10/898,684 (Attorney Docket No. 021496-003000), filed Jul. 23, 2004, which is incorporated herein by reference in its entirety.

Distal end effector 152 of needle grasper apparatus 150 extends from a lumen 21 of overtube 20. The distal end effector comprises grasping element 154 having opposed jaws 156 and 158. Jaw 158 further comprises needle 159. The jaws of grasping element 154 may be approximated, e.g., for grasping items between the jaws and/or for puncturing through tissue via needle 159. In FIG. 12A, end effector 152 has penetrated through the walls of jejunum J and stomach S, e.g., via the needle while jaws 156 and 158 are approximated. The jaws then have been opened to facilitate grasping of suture 63.

Element 154 grasps the suture and pulls anchor 62 out of overtube 20. End effector 152 then is withdrawn from the stomach into the jejunum, as in FIG. 12B. Suture 63 may be utilized to cinch anchor 62, e.g., for formation of an ostomy via pressure necrosis. The suture optionally also may be connected to previously described intestinal anchor 64.

With reference to FIG. 13, the disposition of the securing element and needle/grasping apparatus may be reversed. In FIG. 13A, end effector 152 of apparatus 150; extended through side port 22 of overtube 20 within stomach S, then through the walls of the stomach and intestine into the jejunum; has pulled intestinal anchor 64 out of lumen 21 of overtube 20 within the patient's jejunum J. End effector 152 grasps suture 63 and then is withdrawn to the patient's stomach S, as in FIG. 13B.

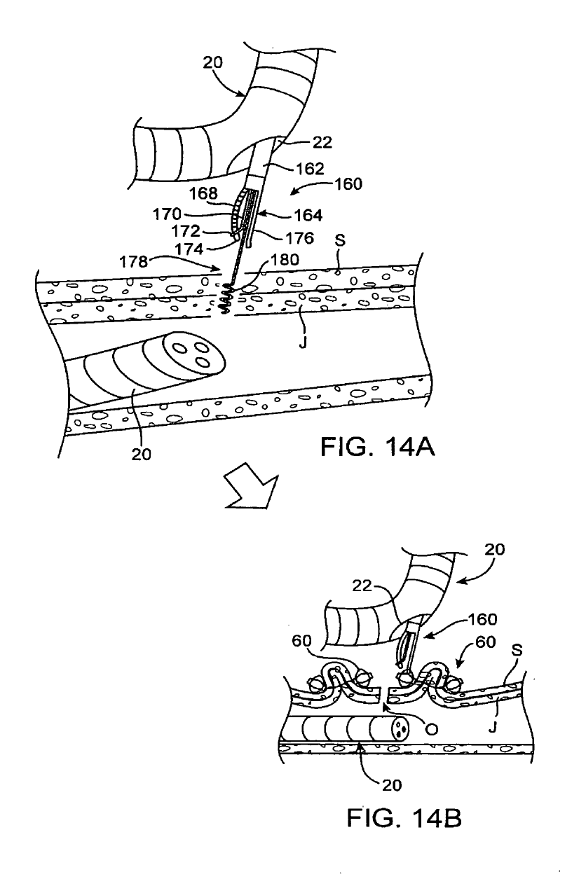

Referring now to FIG. 14, overtube 20 may be used in combination with apparatus for forming and securing tissue folds, e.g., with exemplary securing elements described herein, to secure a patient's stomach to the patient's intestine at a desired location. Tissue plication assembly 160 optionally also may comprise gastroplasty apparatus 110 described herein. Applicant previously has described exemplary plication assemblies, for example, in co-pending U.S. patent application Ser. No. 10/734,562 filed Dec. 12, 2003, and U.S. patent application Ser. No. 10/840,950, filed May 7, 2004, both of which previously have been incorporated herein by reference in their entireties.

FIG. 14A provides an illustrative side view of tissue plication assembly 160 as it extends from side port 22 of overtube 20. Plication assembly 160 generally comprises a catheter or tubular body 162 which may be configured to be sufficiently flexible for advancement into a body lumen, e.g., transorally, percutaneously, laparoscopically, etc., through overtube 20. Tubular body 162 may be configured to be torqueable through various methods, e.g., utilizing a braided tubular construction, such that when a proximal handle (not shown) is manipulated and rotated by a practitioner from outside the body, the torquing force is transmitted along body 162 such that the distal end of body 162 is rotated in a corresponding manner.

Tissue manipulation assembly 164 is located at the distal end of tubular body 162 and is generally used to contact, form and secure tissue plications. Launch tube 168 extends from the distal end of body 162 and in-between the arms of upper extension member or bail 170. Lower extension member or bail 176 may similarly extend from the distal end of body 162 in a longitudinal direction substantially parallel to upper bail 170. Upper bail 170 and lower bail 176 need not be completely parallel so long as an open space between upper bail 170 and lower bail 176 is of sufficient magnitude to accommodate the drawing of several layers of tissue between the two members to form tissue plications. Launch tube 168 may define launch tube opening 174 for deploying a needle and tissue securing elements across such tissue plications, and may be pivotally connected near or at its distal end via hinge or pivot 172 to the distal end of upper bail 170.

Tissue acquisition member 178 may be an elongate member, e.g., a wire, hypotube, etc., which terminates at tissue grasper 180, in this example a helically-shaped member, configured to be reversibly rotated for advancement into tissue for the purpose of grasping or acquiring a region of tissue to be formed into a plication. Tissue acquisition member 178 may extend distally through body 162 of assembly 160 and distally between upper bail 170 and lower bail 176. Acquisition member 178 may also be translatable and rotatable within body 162 such that tissue grasper 180 is able to translate longitudinally between upper bail 170 and lower bail 176.

Tissue manipulation assembly 164, as seen in FIG. 14A, may be advanced through overtube 20 and out side port 22 into the stomach and positioned adjacent to a region of the walls of stomach S and jejunum J to be plicated and secured to one another. Overtube 20 may be utilized to align and approximate the stomach and jejunum, as described previously. Once tissue manipulation assembly 164 has been desirably positioned, tissue acquisition member 180 may be advanced distally such that tissue acquisition member 180 comes into contact with the tissue wall.

If a helically-shaped acquisition member 180 is utilized, as illustrated in FIG. 14, it may be rotated from its proximal end and advanced distally until the tissue walls of both the stomach and the jejunum have been firmly engaged by acquisition member 180. The grasped tissue then may be pulled proximally between upper 170 and lower bails 176 via acquisition member 180 such that the acquired tissue is drawn into a tissue fold. As will be apparent, alternative acquisition members may be utilized to grasp and proximally pull tissue, such as jawed graspers. A tissue securing element, such as securing element 60, then may be placed across the plicated tissue to secure the plication, e.g., via a needle advance through opening 174 of launch tube 168.

As seen in FIG. 14B, this procedure may be repeated, as desired, at multiple locations. For example, a ring of plicated and secured tissue may be formed (see, e.g., FIGS. 2B and 2C). Ostomy O then optionally may be formed in the center of the ring. Alternatively or additionally, an ostomy may be formed through pressure necrosis.

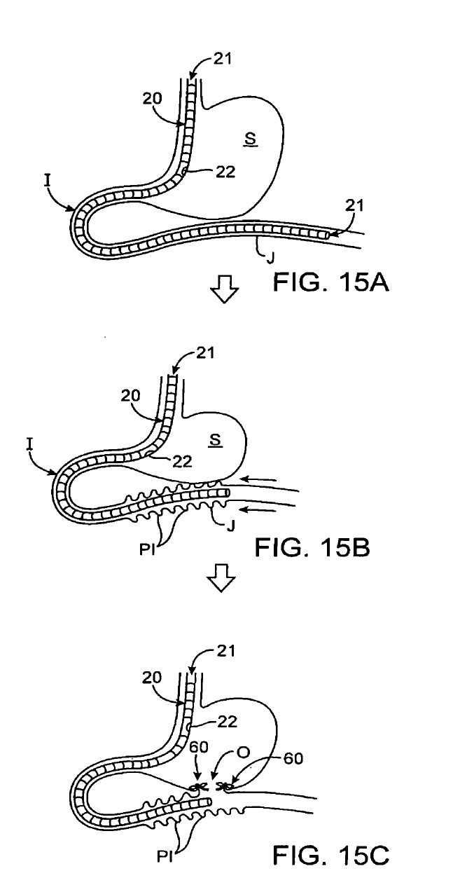

Referring now to FIG. 15, a method of pleating a patient's intestine and conducting a malabsorptive procedure are described. Applicant previously has described methods and apparatus for pleating portions of a patient's gastro-intestinal lumen, for example, in co-pending U.S. patent application Ser. No. 10/746,286 (Attorney Docket No. 021496-000310), filed Dec. 23, 2003, which is incorporated herein by reference in its entirety.

As seen in FIG. 15A, overtube 20 may be advanced through the patient's stomach into the patient's intestine. The distal end of overtube 20 preferably is advanced distal of the position at which jejunum J is in closest proximity or alignment with stomach S. A wall of intestine I then is engaged near the distal outlet of overtube 20, for example, via engagement instrument(s) advanced through a lumen 21 of the overtube, or via suction drawn through such a lumen.

Once the intestine has been engaged, overtube 20 and/or instruments advanced therethrough are withdrawn proximally to bunch up or otherwise form pleats PI within the intestine, as seen in FIG. 15B. The pleated intestine then may be maintained in the pleated configuration by securing the intestine to the patient's stomach distal of, or along, the pleats, as in FIG. 15C. This may, for example, be accomplished utilizing any of the methods and apparatus described previously with respect to FIGS. 13 and 14. Pleating the intestine may reduce a resident time during which food flowing through the gastro-intestinal lumen is in contact with the pleated section of the intestine, thereby reducing absorption of the food as it passes through the intestine.

Gastroenterostomy O optionally may be formed between the stomach and intestine in the vicinity of the secured location to allow food to drain directly from the stomach into the intestine (see, e.g., FIGS. 2B and 2C) distal of the pleated portion of the intestine. In this manner, food may bypass a greater portion of the intestine, as compared to previously described gastroenterostomy procedures. Specifically, food may bypass the additional length of intestine pleated and secured proximal of the ostomy.

Gastroenterostomy procedures described herein illustrate direct securement of the patient's stomach to the patient's intestines at points of ostomy. However, it should be understood that, as an alternative or in addition to their use in performing gastroenterostomy, the methods and apparatus of the present invention may be used to form an ostomy between two portions of the patient's intestines in order to bypass a section of the intestines. Furthermore, as an alternative or in addition to direct securement of the points of ostomy in the patient's gastro-intestinal lumen, an intervening implant, such as a tubular bypass implant, may be secured between the points of ostomy. Bypass implants have been described previously in U.S. patent application Publication No. U.S. 2004/0133147, published Jul. 8, 2004 (U.S. patent application Ser. No. 10/694,149, filed Oct. 27, 2003), which is incorporated herein by reference in its entirety.

Although gastroenterostomy procedures described herein illustratively have been achieved via instruments advanced per-orally and endoluminally through the patient's esophagus, stomach and pylorus into the patient's small intestine, it should be understood that the instruments alternatively may be positioned in the stomach and/or small intestine via a different approach, for example, via a per-anal approach, a laparoscopic approach, a transluminal approach, a transgastric approach, a trans-intestinal approach, a transcolonic approach, a per-pyloric approach, an endo-pyloric approach, a trans-pyloric approach, combinations thereof, etc. Furthermore, gastroenterostomy procedures (as well as other intestinal bypass procedures) optionally may be achieved via instruments advanced transluminally, e.g., per-orally and transgastrically and/or per-anally and transcolonically, to engage and/or approximate, or otherwise mate, the sections of the gastro-intestinal lumen to be joined. An illustrative per-oral, transgastric gastroenterostomy procedure is described, for example, in Applicant's co-pending U.S. patent application Ser. No. 10/______ (Attorney Docket No. 021496-001910US), filed Aug. 11, 2004, which is incorporated herein by reference in its entirety.

Although various illustrative embodiments are described above, it will be evident to one skilled in the art that various changes and modifications are within the scope of the invention. It is intended in the appended claims to cover all such changes and modifications that fall within the true spirit and scope of the invention.

What is claimed is:

1. Apparatus for performing malabsorptive bypass procedures within a patient's gastro-intestinal lumen, the apparatus comprising:
a steerable or shape-lockable overtube configured for advancement into the patient's small intestine;
an anastomosis instrument having proximal and distal regions, and an elongate body extending therebetween, the anastomosis instrument configured for advancement through the overtube into the patient's small intestine to join a portion of the patient's small intestine to the patient's stomach, and to form an ostomy therebetween; and
an alignment mechanism for aligning the portion of the patient's small intestine with the patient's stomach.

2. The apparatus of claim 1, wherein the overtube is configured for advancement into the patient's small intestine via an approach chosen from the group consisting of endoluminal, laparoscopic, per-oral, per-anal, transluminal, transgastric, trans-intestinal, trans-colonic, per-pyloric, endo-pyloric, trans-pyloric and combinations thereof.

3. The apparatus of claim 1, wherein the alignment mechanism is configured for endoluminal or laparoscopic advancement into the patient's stomach.

4. The apparatus of claim 1, wherein the alignment mechanism is chosen from the group consisting of light, telemetry, imaging, sensing, steering, mechanical steering, shape-locking, rigidizing, magnetism and combinations thereof.

5. The apparatus of claim 1, wherein the anastomosis instrument comprises a piercing element for forming the ostomy.

6. The apparatus of claim 1, wherein the anastomosis instrument comprises at least one securing element for joining the portion of the patient's small intestine to the patient's stomach.

7. The apparatus of claim 6, wherein the securing element is configured to form the ostomy through pressure necrosis of joined tissue.

8. The apparatus of claim 7, wherein the securing element comprises a weight configured for disposal within the patient's small intestine to facilitate pressure necrosis.

9. The apparatus of claim 6, wherein the securing element comprises a first portion disposed in the patient's intestine and a second portion disposed in the patient's stomach.

10. The apparatus of claim 9, wherein the first and second portions are magnetic, and wherein the alignment mechanism comprises magnetic attraction between the first and second portions.

11. The apparatus of claim 9, wherein the first and second portions of the securing element comprise central openings through which the ostomy may be formed.

12. The apparatus of claim 1 further comprising an occlusive element for at least partially occluding the patient's pylorus.

13. The apparatus of claim 12, wherein the occlusive element is chosen from the group consisting of hydrogels, adhesives, inflatable balloons, barbed devices, inflammatory agents, chemical irritants, radiofrequency irradiators, heating elements, burning elements, bulking agents, suture, securing elements and combinations thereof.

14. The apparatus of claim 1 further comprising a gastroplasty instrument configured for partitioning a patient's stomach.

15. The apparatus of claim 14, wherein the gastroplasty instrument is configured for advancement through the overtube.

16. The apparatus of claim 1 further comprising a tissue destruction instrument for destroying tissue within the patient's gastrointestinal lumen.

17. The apparatus of claim 16, wherein the tissue destruction instrument is configured for advancement through the overtube.

18. The apparatus of claim 16, wherein the tissue destruction instrument is chosen from the group consisting of radiofrequency instruments, plasma instruments, electrocautery instruments, cryoablation instruments, Argon plasma coagulation instruments, mechanical abrasion instruments, energy instruments and combinations thereof.

19. The apparatus of claim 16, wherein the tissue destruction instrument is configured to destroy tissue within an excluded portion of the patient's stomach.

20. The apparatus of claim 1, further comprising a pleating instrument for pleating tissue within the patient's gastro-intestinal lumen.

21. The apparatus of claim 20, wherein the pleating instrument is configured to pleat a section of the patient's small intestine, and wherein the anastomosis instrument is configured to join the portion of the patient's small intestine to the patient's stomach distal of the pleated section of the patient's small intestine.

22. The apparatus of claim 1, wherein the anastomosis instrument further comprises a plication instrument configured to form and secure tissue folds.

23. The apparatus of claim 1, wherein the overtube is both steerable and shape-lockable.

24. The apparatus of claim 1, wherein the overtube is configured for advancement through the patient's pylorus.

25. The apparatus of claim 6, wherein the securing element is bioabsorbable or bioresorbable.

26. A method for performing malabsorptive bypass procedures within a patient's gastro-intestinal lumen, the method comprising:
steering an overtube into the patient's small intestine;
advancing an anastomosis instrument through the overtube into the patient's small intestine;
aligning the anastomosis instrument with the patient's stomach; and
joining a portion of the patient's small intestine to the patient's stomach with the anastomosis instrument.

27. The method of claim 26, wherein joining a portion of the patient's small intestine to the patient's stomach further comprises forming an ostomy therebetween.

28. The method of claim 27, wherein forming an ostomy further comprises piercing tissue at the joining to form the ostomy.

29. The method of claim 27, wherein forming an ostomy further comprises forming the ostomy through pressure necrosis of tissue at the joining.

30. The method of claim 27 further comprising at least partially occluding the patient's pylorus.

31. The method of claim 27 further comprising performing pyloroplasty to render the patient's pyloric sphincter at least temporarily incompetent.

32. The method of claim 27 further comprising performing gastroplasty within the patient's stomach.

33. The method of claim 27 further comprising locally destroying tissue within the patient's gastro-intestinal lumen.

34. The method of claim 26 further comprising shape-locking or rigidizing the overtube.

35. The method of claim 26, wherein steering the overtube further comprises endoluminally steering the overtube.

36. The method of claim 26, wherein steering the overtube further comprises laparoscopically steering the overtube.

37. The method of claim 26, wherein aligning the anastomosis instrument with the patient's stomach further comprises retroflexing the overtube to a pre-determined configuration.

38. The method of claim 26, wherein joining a portion of the patient's small intestine to the patient's stomach further comprises forming and securing tissue folds encompassing the walls of the patient's small intestine and stomach.

39. The method of claim 26 further comprising pleating a section of the patient's small intestine,
wherein joining a portion of the patient's small intestine to the patient's stomach further comprises joining a portion of the patient's small intestine disposed distal of the pleated section of the small intestine.

40. A method for performing malabsorptive bypass procedures within a patient's gastro-intestinal lumen, the method comprising:
performing gastroenterostomy within the patient's gastro-intestinal lumen;
altering the patient's pylorus;
performing gastroplasty within the patient's stomach; and
destroying tissue within the patient's stomach.

41. The method of claim 40, wherein altering the patient's pylorus comprises at least partially occluding the pylorus.

42. The method of claim 40, wherein altering the patient's pylorus comprises performing pyloroplasty.

43. The method of claim 40 further comprising performing the method endoluminally.

44. The method of claim 40 further comprising performing the method laparoscopically.

45. The method of claim 40 further comprising pleating a portion of the patient's small intestine.
